(19) United States
(12) Patent Application Publication
(10) Pub. No.: US 2011/0016994 A1
(43) Pub. Date: Jan. 27, 2011

(54) PLASTIC ARTICLE AND ITS FABRICATION

(75) Inventor: Gregory Brent WELSH, Beverley Park (AU)

Correspondence Address: PILLSBURY WINTHROP SHAW PITTMAN, LLP, P.O. BOX 10500, MCLEAN, VA, 22102, US

(73) Assignee: SEBEL FURNITURE LTD, Padstow (AU)

(21) Appl. No.: 12/840,582

(22) Filed: Jul. 21, 2010

(30) Foreign Application Priority Data
Jul. 23, 2009 (AU) 2009903434

(51) Int. Cl. G01N 33/44; H01F 1/00; H01B 1/00; H01F 1/04; H01B 1/02

(52) U.S. Cl. 73/866; 252/62.51 R; 252/500; 252/62.55; 252/514; 252/513; 252/512

(57) ABSTRACT

A method of detecting illicit offensive weapons fabricated from plastics articles by felons is disclosed. Electrically conductive and/or magnetically permeable particles are incorporated in the plastics material prior to moulding. The felons are then scanned from time to time using a metal detector. A moulded plastics article and a method of fabricating a moulded plastics article are also disclosed.

### FIELD OF THE INVENTION

The present invention relates to plastic articles and, in particular, to plastic chairs which are supplied to penitentiaries and like penal institutions where felons or other wrong doers are incarcerated.

### BACKGROUND ART

The moulded plastic chair in U.S. Pat. No. 3,874,726 has enjoyed substantial sales to such institutions. The chair is generally manufactured from polypropylene. A problem has arisen in that some inventive inmates of such institutions have fabricated knives or knife like articles from the polypropylene and then secreted the knife like article on their body. Although such felons are from time to time subjected to screening or scanning by means of metal detector, such plastic articles are not detected by this apparatus and therefore the opportunity for wrong doing is not prevented.

### Genesis of the Invention

The genesis of the present invention is the desire to address the abovementioned problem by the provision of various methods and articles which permit such a knife-like weapon to be detected by conventional metal detectors.

### SUMMARY OF THE INVENTION

In accordance with a first aspect of the present invention there is disclosed a method of detecting illicit offensive weapons fabricated from plastics articles by felons and the like, said method comprising the steps of:
(i) obtaining said articles from a plastics material having incorporated therein electrically conductive or magnetically permeable particles which are responsive to remote metal detectors, and
(ii) from time to time remotely scanning said felons using a metal detector.

In accordance with a second aspect of the present invention there is disclosed a moulded plastics article fabricated from a plastics material having incorporated therein electrically conductive or magnetically permeable particles which are responsive to remote metal detection.

In accordance with a third aspect of the present invention there is disclosed a method of fabricating a moulded plastic article, said method comprising the steps of:
(i) incorporating in a moulding plastics compound, prior to moulding same, electrically conductive or magnetically permeable particles which are responsive to remote metal detectors, and
(ii) moulding said article from said compound incorporating said particles.

### BRIEF DESCRIPTION OF THE DRAWINGS

A preferred embodiment of the invention will now be described, by way of example only, with reference to the accompanying drawings in which:

FIG. 1 is a perspective view from the front of the chair of the abovementioned U.S. patent,

FIG. 2 is a perspective view from the rear of the chair of FIG. 1, and

FIG. 3 is a perspective view of a knife like article fabricated from the chair of FIG. 2.

### DETAILED DESCRIPTION

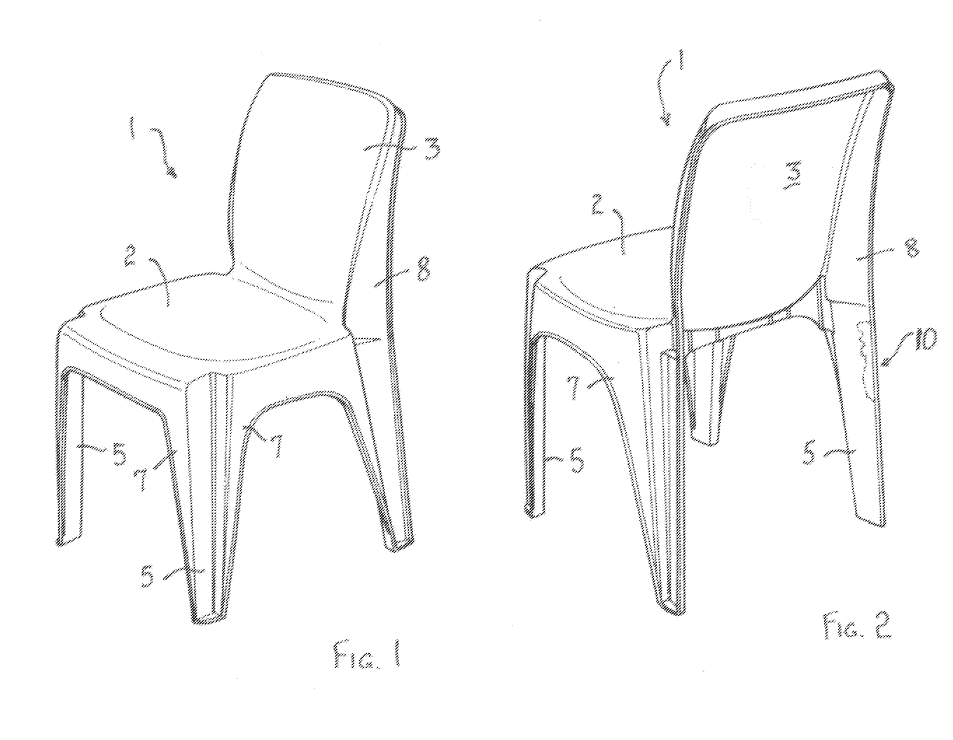

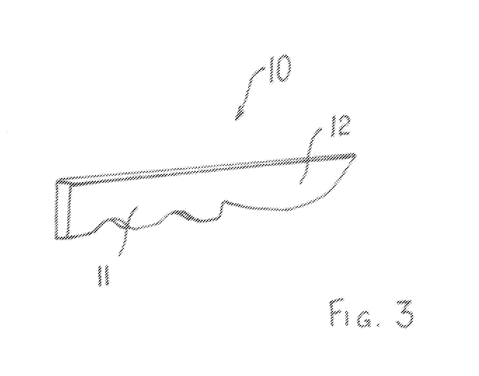

As seen in FIGS. 1 and 2, the chair 1 has a seat 2 and a backrest 3. The chair 1 also has four legs 5. In addition, the chair 1 has three skirts 7 which join adjacent pairs of legs 5, and two webs 8 which extend from the rear legs upwardly to the top of the backrest. The skirts 7 and webs 8 are formed from relatively thin but strong sections of the polypropylene material from which the chair 1 is moulded.

As indicated by dotted lines in FIG. 2, it is possible for one of these thin sections such as a web 8 to have cut from it a knife 10 as illustrated in FIG. 3 having a handle 11 and a blade 12.

Such a knife 10 being formed entirely from plastics material is not able to be detected by a conventional metal detector and so is able to be successfully secreted about the body of the felon or inmate.

Conventional metal detectors work in various ways. One way is to provide a pulse of current to a coil which therefore generates an electromagnetic field which extends outwardly from the coil and then decays. If the electromagnetic field impacts upon an electrically conductive and/or magnetically permeable object, then there is an interaction between the object and the field and this can be detected in a change in the way in which the field decays.

An alternative arrangement, especially in relation to hand held wands, is to provide a coil which forms part of an oscillator circuit. The coil emits an oscillating electromagnetic field. If the wand is moved into proximity to an electrically conductive and/or magnetically permeable article, this results in a change in the inductance of the coil and hence a change in frequency of the oscillation. Again this change in frequency can be detected and the metal object thereby discovered.

In accordance with the preferred embodiment of the present invention, during the fabrication of the chair 1, the plastics compound from which the chair is fabricated is changed by having incorporated therein electrically conductive and/or magnetically permeable particles which respond to conventional metal detectors. In this way, if such a knife 10 is fabricated from such a chair, then the knife 10 responds to the conventional metal detector and its presence can be discovered.

The particles can consist of copper particles, iron particles, steel particles or aluminium particles or lead particles or mixtures thereof. The particles can take the form of powder, flakes, filaments or spheroids (including lead shot, for example), or mixtures thereof. The plastics material from which the chair is fabricated includes polypropylene, and polyvinyl chloride.

It is known to mix metal particles in plastics for various different purposes other than those described above. For example, in U.S. Pat. No. 5,266,246 metal is mixed in with the plastics in order to impart a glossy appearance. In U.S. Pat. No. 4,197,351 metal flakes are mixed in with the plastic powder in order to provide a pigmentation or colour to the resulting plastic. In U.S. Pat. No. 5,177,124 metal particles are mixed with the plastic in a mould and before solidification of the plastic the mould is centrifuged so that the metal particles are forced to the outside of the mould. The moulded article is then brushed or mildly abraded so as to remove a thin plastic coating from the metal particles. In this way the outer surface of the metal particles is burnished so as to create an overall metallic appearance to the moulded article.

None of the abovementioned prior art arrangements is done for the purpose of detecting, using a metal detector, such a knife like article 10 as described above which is fabricated from the chair material.

The foregoing describes only one embodiment of the present invention and modifications, obvious to those skilled in the arts, can be made thereto without departing from the scope of the present invention. For example, the plastic articles from which a weapon can be fabricated potentially include trays, beds, tables and cupboards. The present invention is therefore applicable to such articles which can be blow moulded rather than injection moulded.

After the plastic article is fabricated, it can be shipped or sent to a facility such as a correctional institution or penitentiary. From time to time, felons within the institution can be scanned using a metal detector.

The term “comprising” (and its grammatical variations) as used herein is used in the inclusive sense of “including” or “having” and not in the exclusive sense of “consisting only of”.

1. A method of detecting illicit offensive weapons fabricated from plastics articles by felons and the like, said method comprising the steps of:
(i) obtaining articles from a plastics material having incorporated therein particles which are electrically conductive, or magnetically permeable, or both and which are responsive to remote metal detectors, and
(ii) from time to time remotely scanning said felons using a metal detector.

2. The method as claimed in claim 1 wherein said particles are selected from the group consisting of copper particles, iron particles, steel particles, lead particles and aluminium particles.

3. The method as claimed in claim 2 wherein said particles are selected from the group consisting of powders, flakes, filaments and spheroids.

4. The method as claimed in claim 3 wherein said plastics material is selected form the group consisting of polypropylene, and polyvinylchloride.

5. A moulded plastics article fabricated from a plastics material having incorporated therein particles which are electrically conductive, or magnetically permeable, or both and which are responsive to remote metal detection.

6. The article as claimed in claim 5 wherein said particles are selected from the group consisting of copper particles, iron particles, steel particles, lead particles and aluminium particles

7. The article as claimed in claim 6 wherein said particles are selected from the group consisting of powders, flakes, filaments and spheroids.

8. The article as claimed in claim 7 wherein said plastics material is selected form the group consisting of polypropylene, and polyvinylchloride.

9. The articles as claimed in claim 5 and comprising a chair.

10. A method of fabricating a moulded plastic article, said method comprising the steps of:
(i) incorporating in a moulding plastics compound, prior to moulding same, particles which are electrically conductive, or magnetically permeable, or both, and which are responsive to remote metal detectors, and
(ii) moulding said article from said compound incorporating said particles.

11. The method as claimed in claim 10 wherein said particles are selected from the group consisting of copper particles, iron particles, steel particles, lead particles and aluminium particles

12. The method as claimed in claim 11 wherein said particles are selected from the group consisting of powders, flakes, filaments and spheroids.

13. The method as claimed in claim 12 wherein said plastics material is selected from the group consisting of polypropylene, and polyvinylchloride.
